(19) United States
(12) Patent Application Publication
(10) Pub. No.: US 2026/0007592 A1
(43) Pub. Date: Jan. 8, 2026

(54) MICRONEEDLE PROCESS

(71) Applicant: Corrective Development, LLC, Fair Lawn, NJ (US)

(72) Inventors: Elina KULIYEVA, Brooklyn, NY (US); David OTT, Hawthorne (NY)

(73) Assignee: Corrective Development, LLC, Fair Lawn, NJ (US)

(21) Appl. No.: 19/329,011

(22) Filed: Sep. 15, 2025

Related U.S. Application Data
(63) Continuation of application No. PCT/US2024/020427, filed on Mar. 18, 2024.
(60) Provisional application No. 63/452,708, filed on Mar. 17, 2023.

(51) Int. Cl. A61K 9/00

(52) U.S. Cl. CPC A61K 9/0021; A61K 9/0014

(57) ABSTRACT

The invention relates to a topical cream containing a plethora of small sharp shards of a solid biopolymer (i.e., microneedles) which may or may not contain additional active ingredients or drugs, created in multiple sizes without molds or unusual processing equipment.

### CROSS REFERENCE TO RELATED APPLICATIONS

This application is a continuation application under 35 U.S.C. § 120 of International Application PCT/US2024/020427, filed Mar. 18, 2024, and claims the benefit under 35 U.S.C. § 119(e) of U.S. Provisional Patent Application No. 63/452,708, filed Mar. 17, 2023, the entirety of each of which is hereby incorporated by reference.

### FIELD THE INVENTION

The invention relates to topical treatment and applications of cosmetics to the skin.

### BACKGROUND

Microneedles (MNs) are used extensively, e.g., to apply cosmetics to the skin. Numerous methods are known for producing such microneedles. For example, Sul et al. (U.S. Pat. No. 10,646,702) teaches a method which utilizes molds to produce MNs.

### SUMMARY

The present invention relates to a topical cream containing a plethora of small sharp shards of a solid biopolymer (MNs), which may or may not contain additional active ingredients, such as pharmaceutical drugs, wherein such MNs are created in multiple sizes without molds or unusual processing equipment.

### BRIEF DESCRIPTION OF THE DRAWING

FIG. 1 is a micrograph of 125-250 μm classified microneedles produced by the process of the invention.

### DETAILED DESCRIPTION

The primary purpose of the invention is to more easily create topical microneedle preparations for the use in either treating, moisturizing, or beautifying the skin. The ease of preparation derives from the discovery of a manufacturing method that does not require molding or casting of the polymer. Instead, the creation of microneedles is carried out through a pulverizing/grinding process using readily available milling equipment many cosmetics and topical drug manufacturers already utilize. Once created, the microneedles can then be dispersed in an acceptable anhydrous or minimally hydrated cosmetic base for application to the skin.

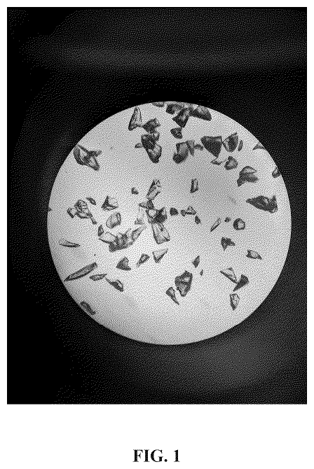

In order to create a functional, cosmetically acceptable skin treatment, the first step is to create microneedles of appropriate size in order to penetrate into the stratum corneum (SC) of the epidermis. The term microneedle in this case refers to a shard of fractured solid non-crosslinked polymer having at least one side that tapers to an acute point (see FIG. 1). Additionally, because the shard is fractured, one or more sides tapering to the point may have a bifaced edge similar to a knife. The point and edge work together to cut and penetrate the SC, the main barrier to absorption of topically applied compounds, especially if polar. Once imbedded in the SC the natural moisture present in the skin will soften and dissolve the polymer making up the microneedle, freeing any entrapped active material and creating a hydrophilic channel other polar active material can move through, enhancing delivery to the lower layers of living skin.

The microneedles of the invention are created as follows. First, a suitable amount of purified water is placed in a vessel equipped with a stirring mechanism. Active ingredients, if included, are dissolved into this water. The actives should be water soluble or able to be solubilized with minimal surfactants or cosolvents. The active ingredient should be included at a low concentration and chosen so as to not interfere with the drying or packing of the polymer which constitutes the microneedles. Peptides and vitamins fill the active role well. Once the active material is fully dissolved the polymer is added and dissolved fully. (Alternatively, the polymer can be dissolved prior to the active ingredient. However, this may make it difficult to fully dissolve the active material.) The polymer should be as concentrated as a pourable viscosity allows. Examples of suitable polymers are but not limited to hyaluronates (native, crosslinked, or alkyl-modified), chitins, oligosaccharides (fructans, beta-glucans), polypeptides (polylysine, collagen peptides), modified cellulose (ethylcellulose, hydroxyethylcellulose), and gums such as gum arabic. More than one polymer, molecular weight, or additional small molecules such as sugars, betaine (trimethylglycine), salts, or amino acids may be used to aid the properties of the microneedles such as fracturability. By sugar is meant mono and disaccharides such as glucose, fructose, lactose, trehalose, sucrose and galactose. By salt is meant any inorganic ionic compound such as sodium chloride, potassium bicarbonate, and magnesium sulfate. At this point 10-40% wt/wt of a lower alcohol should be added to the solution to aid in microbial preservation during the drying period. By lower alcohol is meant a volatile low molecular weight alcohol such as ethanol or isopropanol. Care must be taken, as by watching for reduction in clarity of the solution, to assure that the alcohol does not cause the polymer to come out of solution.

Once the polymer solution has been prepared, it is poured onto a smooth, inert, non-porous tray to a thickness of about 5-10 mm. The tray is placed in a clean environment and allowed to dry slowly at room temperature at a relative humidity of 25-50%. After 24-72 hours the solution will have dried to a tough plastic solid film, and be entirely clear. At this point the tray is moved to an incubator and heated to 40-50° C. for an additional 24 hours to complete the drying process. Once fully dry, the polymer material should have the appearance of a clear brittle plastic, and when crushed breaks into sharp shards.

When fully dried the polymer sheet can be stored in airtight containers until further processing or be processed immediately. To complete the making of the microneedles, the polymer sheet is milled and classified (i.e., dividing the particles by size) as rapidly as possible. Special care must be taken such that the particles do not tumble excessively once reduced to the targeted particle size. Excessive tumbling will round the edges and points, leading to ineffective particles.

Once the microneedle particles are manufactured, they can be stored in airtight containers with a desiccant until use. To use the particles in a product, they can be dispersed into an anhydrous base as simple as a petroleum ointment base or as complex as a multiple emulsion. The choice of base and its technical attributes may easily be determined by one skilled in the art of topical drugs and cosmetic formulations.

The invention is further defined by reference to the following examples, which are intended to be illustrative and not limiting.

### EXAMPLE 1

Purified water for injection (WFI, Intermountain Life Sciences) was charged to a vessel at 55% (weight/weight) of the solution batch weight. Next, Acetyl Nonapeptide-5 (ABG lab) was added at 0.01% and dissolved. Once the peptide dissolved, 25% of hyaluronidase hydrolysed hyaluronic acid with a very low molecular weight (MiniHA, Bloomage Freda) was added in multiple portions slowly. Low molecular weight aids in the drying and brittleness of the film. High molecular weight hyaluronic acid or sodium hyaluronate, >1.8 million daltons results in a tough flexible film after drying. The flexibility of the film makes it much more difficult to reduce the particle size while maintaining points and edges. The solution was stirred until the MiniHA was fully dissolved and the solution was completely clear. When clear, 19.9% wt/wt ethanol was added (i.e., a quantity sufficient to reach 100% of the formulation). The batch remained clear or had the slightest haze. (If the batch becomes more than slightly hazy WFI is added until clarity is restored.) Ethanol is preferred to isopropanol because it has less effect on the solubility of sodium hyaluronate. The fully prepared solution was poured into cleaned and sanitized stainless steel pans at a depth of 8 mm. The exact depth of the solution is not important and can be adjusted based on the desired size of microneedles to be manufactured. The described formulation dried to approximately 12. 5% of the depth of the pour resulting in a film about 1 mm thick. Consistency of pour depth between multiple pans, when used, should be maintained for uniformity and drying time. The poured pans were placed onto a clean bench and allowed to dry for 48 hours at controlled room temperature. After 48 hours the film was checked. The film was dry to the touch, flexible, and completely transparent. Tacky film should be allowed to continue to dry at controlled room temperature until it is dry to the touch and can peel easily from the stainless steel pan without stickiness on the underside. Once the film reached this state, the pans were transferred to an incubator set at 40-50° C. for another 24 hours to complete drying. At this point the film resembles a brittle plastic similar to polystyrene or polycarbonate and about 1 mm thick. At this point any portion of the film that has dried too rapidly to create the required amorphous glass state is evident by appearance. Too rapidly dried film will be opaque, rough, and crumbly. This material can easily be reprocessed by dissolving in WFI and ethanol and repeating the drying process. The film was then placed into a KM-tech Blade Mill milling machine and the particle size was reduced to between 0.1 and 1 mm measured by the particle's largest dimension, as determined by classification screens. Smaller sized batches may be ground in a Waring type blender and manually screened. The size is determined by choice of base and expected product properties.

### EXAMPLE 2

In the following formulations, a MN particle size of <300 μm on the largest dimension was used. 300 μm was chosen because there is tactile feedback to the user that the microneedles are present but were still small enough to not cause pain. 300 μm is significantly larger than is required to penetrate into the SC, which varies from 10 to 30 μm in thickness. Another determination on microneedle size is how quickly the particles dissolve on the skin, because smaller size equals more rapid dissolution. Once the film was produced by drying, and especially once the needles were produced using a Waring blender, care was taken to avoid moisture or excessive relative humidity. Sodium hyaluronate is hygroscopic and the microneedles can become soft or melt if exposed. Prepared microneedles were then dispersed with sweep mixing into the formulations of Formula 1. All the listed commercial ingredients were blended together with simple mixing to create the finished product, which was then filled for use into airless components (i.e., cosmetic containers with a pump and sealed chamber that decreases in particle size via air pressure as the pump evacuates the product).

Formula 1

| | | | Quantity |
|---|---|---|---|
| Vendor | Trade Name | INCI Name | (Wt/Wt %) |
| Resolute Oil | Pionier Sterling | Petrolatum | 76.100000 |
| | White USP | | |
| Alzo | NuLastic Soft BLS | Isodecyl Isononanoate, C4-24 | 15.000000 |
| | | Alkyl | |
| | | Dimethicone/Divinyldimethicone | |
| | | Crosspolymer, | |
| | | Polydiethyleneglycol | |
| | | Adipate/IPDI Copolymer | |
| Centerchem | Neossance | Squalane | 5.000000 |
| Seppic | Sepilift DPHP | Dipamitoyl Hydroxyproline | 1.000000 |
| Lebermuth | Peppermint Yakima | Fragrance (Parfum) | 0.250000 |
| | redistilled | | |
| Spectrum | Allantoin USP | Allantoin | 0.100000 |
| McKinnley | L-hexuron | o-Ethyl Ascorbate | 0.500000 |
| ABG | Sodium Hyaluronate | Sodium Hyaluronate, acetyl sh- | 1.000000 |
| | Microneedles | Nonapeptide-5 | |
| Barnet | Lavendox | Caprylic/Capric Triglyceride, | 1.000000 |
| | | Lavandula stoechas Extract. | |
| Sabinsa | Green Tea Extract | Camellia Sinensis Extract | 0.050000 |
| | C.G. | | |

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention. Furthermore, the foregoing describes the invention in terms of embodiments foreseen by the inventor for which an enabling description was available, notwithstanding that insubstantial modifications of the invention, not presently foreseen, may nonetheless represent equivalents thereto.

What is claimed is:

1. A process for producing microneedles which comprises: a) dissolving a polymer in water; b) transferring the solution of step a) into a tray; c) drying said solution to produce a solid film; and d) milling said film to produce microneedles wherein said polymer is selected from the group consisting of hyaluronates, chitins, oligosaccharides, polypeptides, celluloses, and polylactides.

2. A process of claim 1 wherein the water of step a) contains an active ingredient selected from the group consisting of water-soluble peptides, vitamins, sugars, betaine, salts, amino acids, or combinations thereof.

3. A process of clam 1 wherein the water of step a) contains a lower alcohol.

4. A process of claim 1 wherein the microneedles have a particle size of between 0.1 and 1 mm measured by their largest dimension.

5. A process of claim 1 further comprising dispersing the microneedles of step d) into an anhydrous or minimally hydrated cosmetic base.
